(19) United States
(12) Patent Application Publication
(10) Pub. No.: US 2012/0143236 A1
(43) Pub. Date: Jun. 7, 2012

(54) DEVICE FOR REFLEX CORRECTION OF FUNCTIONAL DISORDERS OF THE ORGANISM

(76) Inventor: Valrey Gennadievich Muzhikov, St. Petersberg (RU)

(21) Appl. No.: 13/261,046

(22) PCT Filed: May 21, 2010
(86) PCT No.: PCT/RU2010/000276

§ 371 Date: Feb. 21, 2012

(30) Foreign Application Priority Data
May 28, 2009 (RU) 2009120325

(51) Int. Cl. A61B 17/34

(52) U.S. Cl. 606/189

(57) ABSTRACT

A device for monitoring and correction of a functional condition of a human body on the basis of measurement of thresholds of temperature painful sensitivity (TPS) of acupuncture points (AP). The device includes a diagnostics and treatment module, a control and communication block and a server, connected by bi-directional data channels. The module includes a handle with an autonomous power unit and a means of reception/transmission. The module is supplied by an element of thermal influence on AP in the form of an infrared LED connected to a current generator, a radiation indicator, a switch, inputs/outputs and a microcontroller. The control and communication includes a mobile phone or a computer of the patient with a related function of data reception/transmission, display of the data and control of the module, by means of inputting of a resident program. The handle serves as the peripheral indicator of a condition and the device of influence with the low price for the patient, and all processing of the address individual information of each patient is assigned to the server.

The invention relates to medical equipment, in particular, to reflexology, and can be used for monitoring and correction of functional state of the human body by measuring thresholds of temperature pain sensitivity (TS) of acupuncture points (AP) “entrance/exit” of the main channels.

There are devices for monitoring the functional state of a single organ or physiological system of the organism, based on measuring of various indicators, such as electrical biosignals, the potential of the electrocardiogram, microcurrents from AP main channel, the electromagnetic radiation of human and the thresholds of temperature pain sensitivity (TS) of acupuncture points (AP) “input/exit” the main channels.

The microcurrents are measured in turn at the representative AP on the human body by the sensor in the form of passive and active electrodes of the device for complex estimation of the state of human health according to the patent (RU 2215471 C2), and the measured data are feed into a computer through autonomous block of transformation and amplification. Measurement of biological currents is accomplished in a dialogue with the computer, the analysis in which formed as the detailed characteristics of the functional state of organs and body systems with their visualization. The disadvantage of the device on the patent RU 2215471 is the lack of channels of measured data from the autonomous blocks remote patients to the server, which reduces the efficiency of computing resources.

There are a system for diagnosing the cardiovascular system according to the patent for utility model (RU 48261 U1, IPC A61V 5/02, op. Oct. 10, 2005), which contains at least one stand-alone portable system for collecting kardiometric data, connections, transmission channels computer network with data processing system. Each portable data collection kardiometric system contains a sensor. It includes electrodes that are connected through multi-channel amplifier to inputs of a multichannel device analog to digital conversion. It is the output of the sensor. Output of the sensor is connected to the microprocessor, which is connected to display unit information and communication block with the computer network. Processor, display and GPRS-modem of mobile phone can be used as the microprocessor unit and display unit with computer network. Data processing system is configured to collect automatic or automated processing kardiometric data from two or more portable kardiometric data collection systems. Kardiometric portable data collection system is configured to display derived from the data processing system of processing results. The system diagnostics of the human cardiovascular system of the stated patent provides more efficient use of computing resources processing block (server) by ensuring the processing of data from two or more portable kardiometric data collection systems, but it doesn't solve the problem of correcting the functional state of the organism.

There is a personal system for monitoring the state of the patent for the invention (RU 2285442 C2), which refers to a system for monitoring glucose levels of patients with diabetes mellitus. The system contains input devices consulting unit and the device output. The device input is designed for data entry history of current and/or prospective data relating to the state in consultation unit. Consulting unit is designed for receiving and storing medical history of current and/or prospective data relating to the status received from the input device data, and processing of those data relating to a condition for the development of therapeutic recommendations. The output device is designed to receive medical advice from an advisory devices and connection with the patient. The common device provides the ability to data input related to the level of glucose in patients with diabetes status, processes information in the advisory unit to provide medical advice in the form of advice and if it's necessary it solves the problem of correcting blood glucose levels. However, this device doesn't include biorhythms changes in glucose levels in patients which reduces the clinical effect of developed therapeutic recommendations.

The termodiagnostic test Akabane is used widely in the modern reflexology to monitor the state of an organism, which is based on thermal effects on the balanced end AP the “entrance/exit” the main channels (meridians), to measure their temperature thresholds of pain sensitivity, and further evaluation of the data (Portnoy F. G. Electropuncture reflexology.—Riga.: Zinante, 1998.—P. 103. Muzhikov V. G. Theory and Practice termopunktural channel diagnostics and treatment.—St. Petersburg.: “ Petrovsky Fund” Ltd, 2000 .—P. 27-37). The following commonly used designations are located in the phalanges of hands and feet of symmetric terminal AP main channels (see FIG. 5), which are used in the description of the invention: LU11—lung; L11—colon; HC9—pericardium, TH1—triple heater; Ht9—heart; SI1—small intestine; SP1—spleen; Liv1—liver; St45—stomach; GB44—gallbladder; Ki1—kidney; BI67—bladder. Right and left branch (side) of channel are represented by indices d and s, respectively. Further description uses only the name of AP, a number of points omitted.

Traditionally it is thought that virtually healthy human sensitivity of left and right branches of the channel should be the same. If there is pathology the left and right channels have different branches of the TS, the degree of asymmetry between them increases in proportion to severity of disease. The expression refers to the functional canal breach of its condition when is a significant asymmetry (30-40%) among its branches, which indicates the presence of functional abnormalities in the organ or system associated with this channel. By changing the TS by the thermal effects on AP channel can carry reflex correction of functional disorders of organs and body systems.

There are some devices for reflex correction of functional disorders of the cardiovascular system, such as arterial dysfunction or cardiac arrhythmia (RU 2152777 C 1, RU 2149625 C1). TS channels Akabane test in the dynamics of observations at different values of arterial pressure (RU 2152777) or the number of heart contractions (RU 2149625) are estimated by these devices. The channels with the biggest influence on the normalization of the controlled parameter are determined according to the correlation and regression analysis of the TS channels and then AP identified channels are influenced by modulated infrared radiation to normalize blood pressure (the number of heart contractions).

TS channel changes rhythmically under the influence of physiological rhythms, which are accepted as working cycles of functioning cells, organs and body systems with periods ranging from milliseconds to minutes. At a maximum TS channel and this organ (physiological system), the function which it reflects, is in a state of functional impairment (hypofunction channel). If the TS channel is low, it indicates the presence of functional redundancy (hyperfunction canal and associated organ).

A disadvantage of devices for patent RU 2152777 and RU 2149625 is the lack of synchronization with the rhythmic impact of the change of TS channels under the influence of physiological rhythms. As a result exposure time to achieve clinically beneficial results increases.

Reflex correction of the functional state based on the identification of rhythmic changes TS channels and reflex action in moments of the projected location in the channel hypo function is solved in the device for reflex correction of functional disorders of the body of a utility model patent (RU 64068 U1, IPC A61N 39/00, published 27 Jun. 2007), which is chosen as the prototype.

A device for reflex correction of functional disorders of the body according to the patent RU 64068 U1 contains the IR-probe which is connected with the controlled current generator, multifunction displays, control buttons and a microcontroller that includes a network connected to the microprocessor bus, program block, block of data memory and a timer/counter. Controlled current source IR-probe, start/stop switch and form a microcontroller module diagnosis and treatment, which provides measurement of the temperature thresholds of pain sensitivity (TS) of acupuncture points (AP) “input/output” channels and main medical reflex effect on AP-modulated infrared light. IR-probe is designed as a handle of insulating material; on the outer surface of it there is a button switch start/stop, but in the end of handle infrared LED is placed. Microcontroller-controlled current source and the remaining elements of the device placed in a device that is used to monitor a patient.

The disadvantage of the device which is taken as a prototype is the redundancy of the equipment of the patient. It causes an increase of the cost of the device, as well as the impossibility of monitoring a large number of patients.

This intention is aimed to solve the problem. The problem is to create a device for reflex correction of functional disorders of the organism as a standalone diagnosis and treatment module which is integrated with communication and control unit and a remote server which processes the received from patients results and issuing recommendations to the reflex action by means of modulated infrared radiation.

The technical result which is achieved by using this intention consists in increasing of the computing resources efficiency, reducing equipment cost and enhancement of the patient's functional capabilities.

The problem of achieving the above-mentioned technical result is solved by the fact that a server and communication and control block are placed into the device for reflex correction of functional disorders of the body that contains the diagnosis and treatment module, which is configured for measuring the temperature thresholds of pain sensitivity (TS) of acupuncture points (AP) “input/output” of main channels and medical reflex correction on the AP-modulated infrared light. The communication and control block is connected by bi-directional data channels with the diagnosis and treatment module and the server, diagnosis and treatment module is provided with power supply, an indicator of radiation and its made with an additional opportunity to transmit/receive data and transfer resident program to the control and communication block; communication and control block is configured to transmit/receive data input of the measured physiological or biochemical parameters, data display and control of diagnosis and treatment module; the server is configured to process data TS AP of each patient by constructing models of the interconnection with the TS AP with physiological or biochemical parameters of an individual organ or physiological system, the allocation of the dominant rhythms of TS channels, their prediction and formulating of treatment recommendations on realizations of influence with formation the picture off the location of AP for therapeutic influent to be displayed in the control and communication block.

The technical result is achieved by the following way:

the mobile phone or computer of the patient with laying on an internal microprocessor, display, control and power transmit/receive functions to receive/send data, input of the measured physiological or biochemical parameters, data display and control of the diagnosis and treatment module by entering the resident program of the diagnosis and treatment module are used as control and communication block; bi-directional data channel between the diagnosis and treatment module and a control block and a wired connection is made and/or wireless such as a system transmit/receive data [symbol] Bluetooth [symbol] ;

the channels of the Internet are used as bi-directional data transmission channel between communication and control block and the server;

diagnosis and treatment module has an element of the thermal effect on the AP in the form of infrared (IR) LED and the indicator of radiation that are connected to the controlled current generator, a microcontroller, that includes a connection to network bus, which is the input/output microcontroller, a microprocessor, a program block, the block of data memory and a timer/counter, power supply, transmit/receive block and input/output block, thus the IR LED anode is connected to the positive bus of controlled power generators, whose minus bus, cathode LED diode and the first contact switch start/stop are connected to the shared bus “land” and the second contact switch start/stop, the entrance of controlled current generator, the output power supply block and input/output block reception/transmission is connected through input/output of the microcontroller;

program block of microcontroller includes two-way connected to the program block bus, which is its input/output, power management block, block of the signal control formation of the current generator, block measuring TS channel, block identification and block of the resident program;

power supply block includes consistently connected the block of a managements charge, accumulator block and block of inclusion of a power whose output is the output of the power supply block, while block of a managements charge is provided with a jack plug for connection to an external power adapter, and block the inclusion of power is provided with power switch with an indicator of inclusion;

diagnosis and treatment module is made in the form of a insulating material handle, in the end of it the IR LED is placed, and the outer surface of the a handle are an indicator light, switch start/stop, switch power, indicator of inclusion, socket for connection an external power adapter and socket for connection a wired communication channel.

The technical result is ensured by the distribution of computational resources required for the functioning of the device, the autonomous diagnosis and treatment module, communication and control unit and the remote server. In the case of using mobile phone or computer of the patient as the control and communication block and the remote server for the primary processing of measurement results of the patient, the module diagnosis and treatment that is used as handles, has a small part of the required hardware and software equipment and has a low cost. Enhancement of the devise functionality is ensured by the remote server to monitor many patients.

The intention is illustrated by the drawings, which depicted:

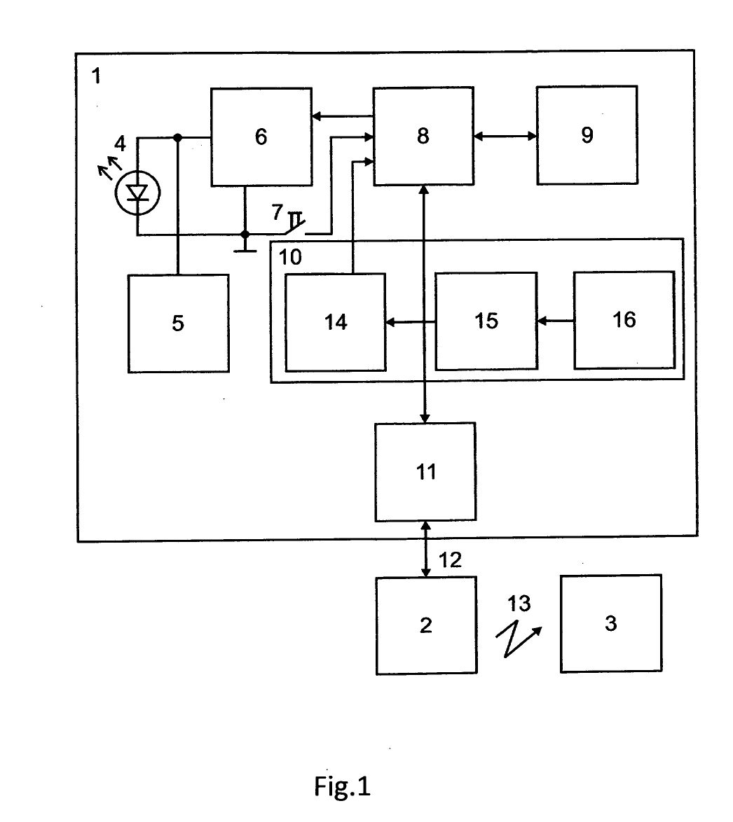

FIG. 1—structural electric circuit of the device;

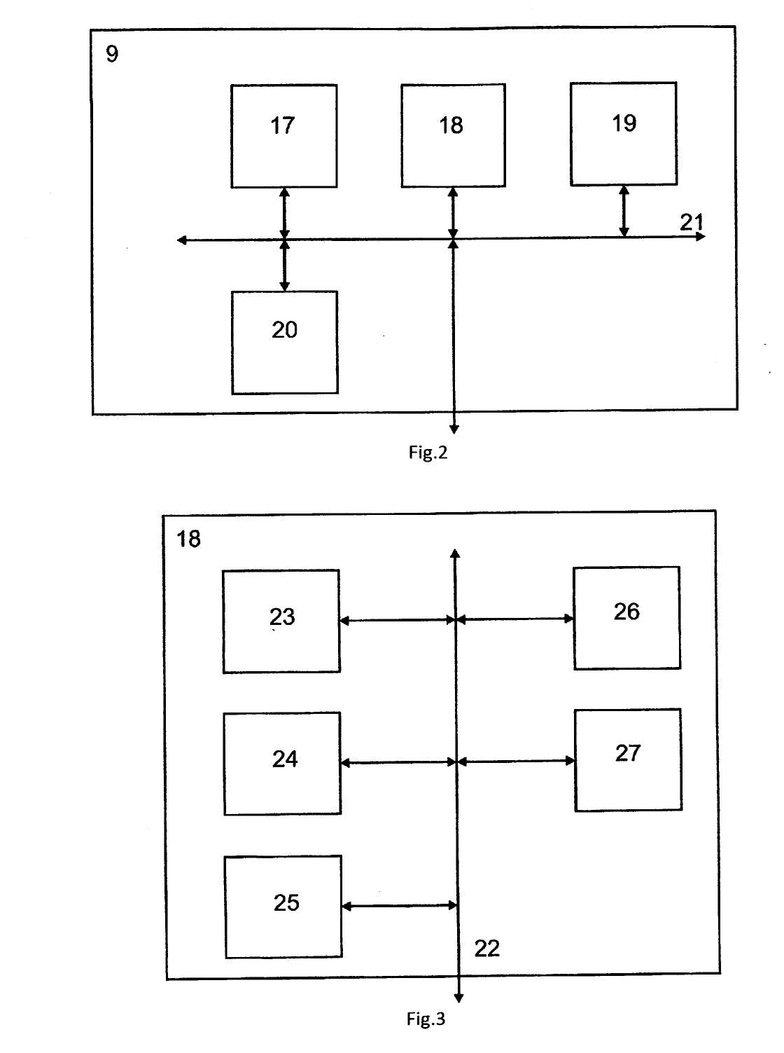

FIG. 2—structural electrical circuit of the microcontroller;

FIG. 3—structural electrical circuit of the program block;

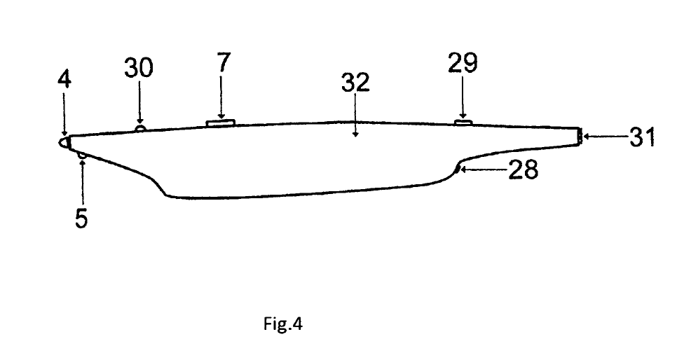

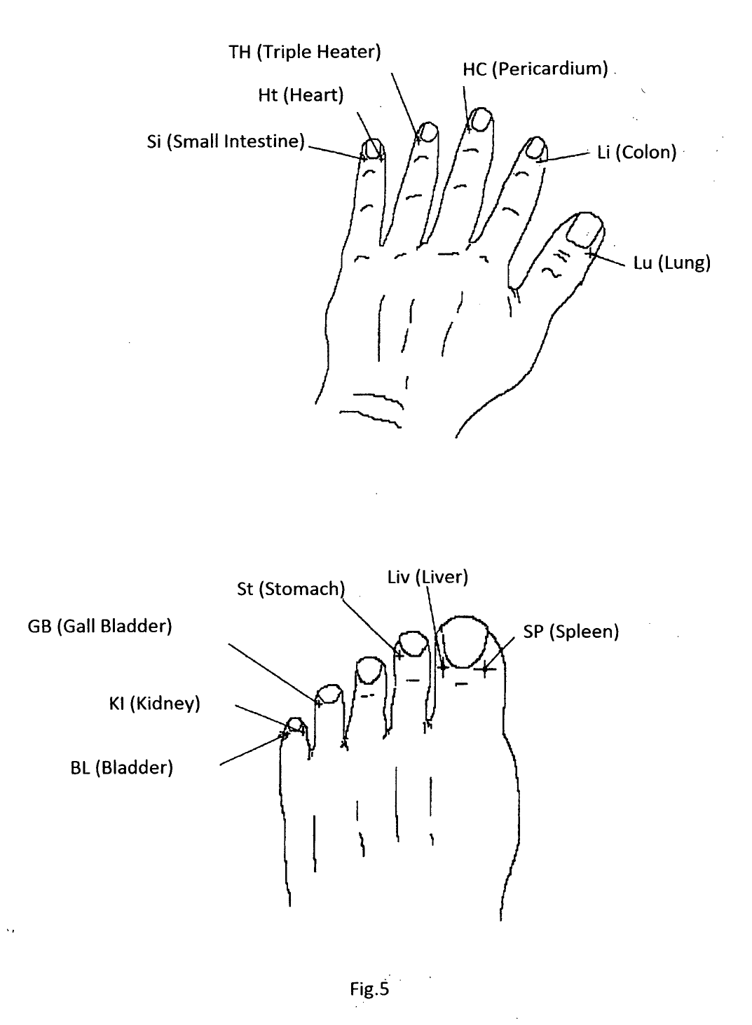

FIG. 4—general view of the handle;

FIG. 5—location of symmetric AP the entrance/exit of the main channels.

The device for reflex correction of functional disorders of the organism consists of three parts: the diagnosis and treatment module 1, control and communication block 2 and server 3 which is connected by channels of communication. The control and communication block 2 is connected through bi-directional channel 12 data transmission to module 1 and bi-directional data transmission channel 13 to the server 3.

The diagnosis and treatment module 1 is made in a form of separate self-powered pen and is intended for testing and therapeutic effects. In fact, it is specialized accessories for medical use to the communication and control block 2.

The mobile phone or computer of the patient with laying on an internal microprocessor, display, control and power transmit/receive functions to receive/send data, input of the measured physiological or biochemical parameters, data display and control of the diagnosis and treatment module by entering the resident program of the diagnosis and treatment module 1 are used as control and communication block 2.

Bi-directional data transmission channel 12 between the diagnosis and treatment module 1 and the control and communication block 2 can be done wired and/or wireless. For example the wireless bi-directional channel 12 of data transmission can be done in the form of transmit/receive [symbol] Bluetooth [symbol] . If the bi-directional data transmission channel 12 is wired, module 1 can be connected to block 2 through USB port and is supplied with connector 31.

As a bi-directional data transmission channel 13 between the control and communication block 2 and the server 3 can be used by any wireless communication channel, such as the Internet.

Server 3 is intended for transmit/receive of data from the patient, their processing and issuance of medical advice.

Module 1 of diagnosis and treatment has an element of the thermal effects on the AP in the form of IR LED 4 and LED 5 light which are connected to the controlled current generator 6, the microcontroller 9, power block 10 and reception/transmission block 11 which are connected through block 8 with entrance/output with entrance/output of the microcontroller block 9. Anode IR LED 4 is connected to the positive bus of controlled current generator 6 whose minus bus and cathode IR LED 4 and the first contact of the switch 7 start/stop are connected to the shared bus “land”. The second contact of the switch 7 start/stop and the entrance of controlled current generator 6 are connected through block 8 input/output to the input/output of the microcontroller 9. The microcontroller 9 includes a microprocessor 17, program block 18, data memory block 19 and timer/counter 20 which are connected to the power bus 21. This bus is the input/output of the microcontroller 9.

As block 2 of control and communications there is used a mobile phone or computer of the patient. The functions connected with receiving/sending data, inputting the measured physiological or biochemical parameters, data displaying and controlling of module 1 responsible for the diagnosis and treatment are laid on an internal microprocessor, display, control parts and power units of transmit/receive. These functions are performed by entering the resident program from the module 1 of diagnosis and treatment.

Bi-directional data transmission channel 12 between the module 1 of diagnosis and treatment and block 2 of control and communication can be done both wired and/or wireless. When the bi-directional channel 12 is performed in a wireless data transmission way, it can be done, for example, in the form of transmit/receive [symbol] Bluetooth [symbol] system. When the bi-directional data transmission channel 12 is performed in a wired way, module 1 may be connected to block 2 via a USB port and is equipped with connector 31.

As a bi-directional data transmission channel 13 between the block 2 of control and communication and the server 3 can be used by any wireless communication channel, such as the Internet.

Server 3 is designed to transmit/receive data from the patient, their processing and issuance of medical advice.

Module 1 of diagnosis and treatment has an element of the thermal effects on the AP in the form of infrared LED 4 and radiation indicator 5 which are connected to the controlled current generator 6, the microcontroller 9, power block 10 and block 11 of reception/transmission, connected via block 8 of entrance/output with block 9 of reception/transmission of the microcontroller. Anode IR LED 4 is connected to the positive bus controlled current generator 6, which minus bus and cathode IR LED 4 and the first contact of the switch 7 Start/Stop are connected to the shared bus “land”. The second contact of the switch 7 Start/Stop and the entrance of controlled current generator 6 are connected through block 8 of input/output with the input/output of the microcontroller 9. The microcontroller 9 includes a microprocessor 17, programs block 18, block 19 of data memory and timer/counter 20, connected to the network bus 21, which is the input/output of the microcontroller 9.

Block 8 of input/output provides a coupling device with a microcontroller 9 via its constituent interfaces of input/output according to the number of mating block.

Programs block 18 of the microcontroller 9 is configured with the possibility to control operating modes, to transfer the controlled current generator 6 from the measuring mode into the mode of influence, to measure the TPS channels, to identify unit 2 of control and communications and to enter residential programs.

Block 18 of microcontroller programs contains block 23 of control modes, block 24 forming the signals for controlling the current generator, block 25 of measuring TPS channels, the identification block 26 and block 27 of resident program, which are connected by means of the two-way communication with the bus of the 22 block of programs, which is its input/output.

Block 10 of the power supply contains consistently connected block 16 of charge control, accumulator battery block 15 and block 14 of turning on power whose output is the output of block 10 of power supply. Block 16 of charge control is equipped with the connector 28 for connection to an external power adapter, and block 14 of turning on power is equipped with the power switch 29 and with the indicator 30 of turning on.

Module 1 of diagnosis and treatment (see FIG. 4) is designed as a handle 32 made of insulating material. At the end of it IR LED 4 is located and on the outer surface of the handle there are located an radiation indicator 5, the button switch 7 start/stop, connector 28 for connecting external power adapter, power switch 29, indicator 30 of start on and plug 31, which is designed to connect the block 11 of receiving/transmitting to the wired communication channel.

Device for reflex correction of functional disorders of the organism works as follows. Charge the accumulator battery block 15 from external power adapter with the charge control unit 16.

By the help of switches handle 29 the power is supplied from the power block 10 to the device 1 of diagnosis and treatment, located in the handle 32. Thus in the block 18 of programs it is run block 26 of identification of the block 2 of control and communications. In further description will assume that as a block 2 it is used a mobile phone of the patient. When joining into the connection in the block 18 of programs there is run block 27 of input of resident program and in the mobile phone there is introduced a resident program. On the display of mobile phone there is the main menu, which has options: “diagnosis”, “transfer data to the server, “receiving data from the server” and “treatment”. At the same time into the phone there is installed a program that allows to make the measurement of the current state of the organism under TPS AP through the option “diagnosis” and to pursue appropriate medical correction through the choice option “treatment”, in case if from the server according to the results of simulation and prediction of the current state of the organism will be sanctioned the present influence.

For estimating the state of the organism there is formed a training set by means of measuring the analyzed indicator of the rate of the organism with its various data in time and simultaneous measurement of temperature thresholds of pain sensitivity (TPS) located in the phalanx of hands and feet of symmetric standard distal acupuncture points (AP) of the “entrance/exit” the main channels.

To measure the TPS channels when accessing the option “diagnosis” on the display, there is alternately shown the position of AP, starting with the first. The measurements are made alone by the patient or with the help of medical personnel. Puts the handle 32 on the first AP right branch of the canal LUd, located at the phalanx of the thumb of his right hand of the patient (see FIG. 5 a), with the direct contact with LEDs 4 with the skin surface. Push button of the switch 7 start/stop. At operation of the switch 7 there is formed the launching strobe/impulse, which activates the controlled current generator 6 in the measurement mode and activates a timer/counter 20 of the microcontroller 9. Meanwhile, there is activated the indicator 5, made, for example, in the form of LEDs in the visible wavelengths, which signals the presence of infrared radiation that is necessary to ensure the patient's safety.

In the “diagnosis” mode from a controlled current generator 6 there is supplied to the LED 4, for example, the sequence of pulses with a repetition period 1 sec, with the pulse duration ranging from 0.7 to 0.9 sec and a current value, able to provide infrared radiation light-emitting diode 12 with a power density pulse of 200 mW/cm2. Source of heat is the infrared light-emitting diode 4, which lens is a hemisphere with a radius of about 1.5 mm, which allows you to measure the TPS AP from the skin surface, corresponding in area to the morphological substrate of AP. The length of the radiated wave can be in the range from 780 to 1600 nm, the same as the “transfer water window”, and the optimal permeability of the skin and tissues (see patent RU N2122208, MPK 6 G01N 33/49, A61V 5/00; publicity 20 Nov. 1998). It is preferred to use as a light-emitting diode 4 IR LED type 3AL107B with the length of the radiated wave 920 nm, which showed reliable results. The power density is selected from the conditions of the dynamic range of changes of TPS channels in different states, ranging from 2 to 100 seconds, at an average threshold of 5 to 10 seconds. This condition is satisfied by the power density of infrared radiation in a pulse of 200 mW/cm2. The impact in a pulsed mode with a repetition period 1 sec and a pulse duration ranging from 0.7 to 0.9 sec is chosen empirically based on the set of observations and gives a more precise threshold for perception of pain.

At occurrence of the first sensations of a patient's pain the button switch 7 should be pressed again. The resulting stopping strobe/impulse turns off the current generator 6 and stops the timer/counter 20. The time interval from the start of irradiation AP to the first pain, measured by the timer/counter 20, at a constant radiated power density is proportional to the energy radiated by an LED 4, and corresponds to the physiological threshold of thermal pain sensitivity (TPS), in this case of the right branch canal of the lung (LUd).

The measurements data of TPS of all AP through the block 11 of receiving/transmission are received in block 2 of control and communications throughout the handle 32. After testing the results of diagnostic may appear in the form of summary tables and additional illustrating graphs. Then the measurement data are transmitted to the server 3, where the patient is identified, for example, through a telephone SIM-card.

Measurement TPS data of each patient are processed on the server 3. According to the received training sorting there the significant channels are defined and there a model of the relationship between the analyzed physiological or biochemical indicators of the organism and the values of TPS significant channels are formed. Then, from the measurements of TPS significant channels, the dominant biorhythms of TPS significant channels are allocated from the training sorting, extrapolated for the future and developed treatment guidelines for conducting influents. Also there is formed a drawing of the location of AP for therapeutic influents, which is transmitted for display on a mobile phone. In the mode “treatment” for mobile phone on the display there is sequentially displayed picture of the position of AP for therapeutic influents.

For the treatment the controlled current generator 6 is transferred into the mode of action. In this mode, the current generator 6 generates, for example, the sequence of impulses with variable frequency rate from 28±4 Hz, with an impulse duration equal to 0.5 from the period of repetition, and the amount of current that provides 4 LED infrared radiation with a wavelength in the range of 780-1400 nm and power density varying linearly during the time of influence from 200 mW/cm2 up to 300 mW/cm2. To start the influence handle 32 should be placed on the specified AP and the button switch 7 should be pressed. Influence on AP is performed before the first pain indications of the patient (before achievement of the TPS channel). Influence is stopped by pressing the button switch 7. Then the influence is performed on the subsequent specified AP of the patient, which provides a reflex correction of functional disorders of the organs.

Construction of the device in the form of modular design construction increases the efficiency of usage of the computing resources, reduces the cost of the equipment of the patient and extends the functionality of the device. Every patient has a mobile phone or a computer with their help the basic functions of the reception/transmission and data display are performed. The handle serves as a peripheral sensor of a condition and the device of influence with the low price for the user, and all the processing of address individual information of each patient is assigned to the server.

1-10. (canceled)

11. A device for reflex correction of functional disorders of the body, the device comprising:
a diagnosis and treatment module configured to measure a plurality of temperature thresholds of pain sensitivity (TPS) of acupuncture points (AP) input/output of major channels and medical reflex correction on AP-modulated infrared light;
a server; and
a communication and control block connected by at least one bi-directional data transmission channel with the diagnosis and treatment module and the server,
the diagnosis and treatment module receives/sends data, inputs measured physiological or biochemical parameters, and displays data,
the server is configured to process TPS AP data of a patient by constructing a model of a relationship of the TPS AP data with physiological or biochemical parameters of an individual organ or physiological system, and an allocation of dominant rhythms of TPS channels, and
the communication and control block configured to display the data.

12. The device of claim 11, wherein the diagnosis and treatment module includes a power unit and a radiation indicator.

13. The device of claim 11, wherein the dominant rhythms are identical and serve to identify treatment recommendations with the simultaneous forming of a picture of AP locations.

14. The device of claim 11, wherein the control and communication block includes a mobile phone or a computer of the patient for the diagnosis and treatment that are stored on an internal microprocessor by entering a resident program from the diagnosis and treatment module.

15. The device of claim 11, wherein the at least one bi-directional data transmission channel between the diagnosis and treatment module and the control and communication block is wired or wireless.

16. The device of claim 11, wherein the at least one bi-directional data transmission channel between the control and communication block and the server includes a wireless communication channel.

17. The device of claim 11, wherein the diagnosis and treatment module includes an element of thermal effects on the AP in a form of infrared LED and a radiation indicator that are connected to a controlled current generator, a microcontroller, a power block and a block of reception/transmission.

18. The device of claim 11, further comprising a block of microcontroller programs comprising a block of control modes, a block of forming the signals for controlling the current generator, a block of measuring TPS channels, an identification block and a block of resident program, which are connected by means of a two-way communication with a bus of the block of programs.

19. The device of claim 11, further comprising a power supply comprising a connected means of charge control, an accumulator battery and a means of turning on power whose output is the output of power supply.

20. The device of claim 11, wherein the diagnosis and treatment module is comprised of a handle of insulating material, and further wherein at one end is located an IR LED and on an outer surface of the handle is a radiation indicator, a button switch to start/stop, a connector for connecting an external power adapter, a power switch, an indicator of an on condition and a plug to connect to a wired communication channel.
